(19) United States
(12) Patent Application Publication
(10) Pub. No.: US 2021/0378967 A1
(43) Pub. Date: Dec. 9, 2021

(54) HARD-PRESSED SCORED SPLITTABLE MARIJUANA TABLETS

(71) Applicant: Columbia Care LLC, New York, NY (US)

(72) Inventors: Aaron Michael Dely, Lighthouse Point, FL (US); Donald Matthew Mordas, Simpsonville, SC (US)

(21) Appl. No.: 16/975,302

(22) PCT Filed: Feb. 21, 2019
(86) PCT No.: PCT/US2019/019015

§ 371 Date: Aug. 24, 2020

Related U.S. Application Data
(60) Provisional application No. 62/634,623, filed on Feb. 23, 2018.

(51) Int. Cl. A61K 9/20; A61K 36/185; A61K 31/352; A61K 31/05

(52) U.S. Cl. CPC A61K 9/2072; A61K 36/185; A61K 31/352; A61K 2236/37; A61K 9/2095; A61K 9/2059; A61K 31/05

(57) ABSTRACT

The invention relates to cannabinoid tablet compositions and methods associated therewith. Specifically, the invention relates to a hard-pressed, scored cannabinoid tablet that can be easily divided to provide multiple doses.

### FIELD OF THE INVENTION

The invention relates to cannabinoid tablet compositions and methods associated therewith. Specifically, the invention relates to a hard-pressed, scored cannabinoid tablet that can be easily divided to provide multiple doses.

### BACKGROUND OF THE INVENTION

The medicinal and psychoactive properties of the Cannabis plant have been known for centuries. While its use has been illegal in many countries, there is a growing population lobbying for legalization of its use, especially for medicinal purposes.

Cannabis is believed to provide benefits in the treatment of multiple disorders with safer and fewer serious side effects than most prescription drugs currently used as antiemetics, muscle relaxants, hypnotics, and analgesics. A disadvantage in treating patients with Cannabis is the psychoactive effect, especially in “naive” Cannabis users. Furthermore, there have been reports of unpleasant reactions to Cannabis, such as anxiety, panic, or hallucinations. It is believed that the undesirable side effects are most commonly associated with higher doses of Cannabis and are related to the difficulty in controlling the dosage when the drug is smoked or eaten in Cannabis-enriched confectionaries.

Cannabis has also been used to treat the symptoms in patients suffering from serious medical conditions. For example, Cannabis has been used to alleviate symptoms associated with cancer, anorexia, AIDS, chronic pain, muscle spasticity, glaucoma, arthritis, migraine, and many other illnesses. Cannabis is recognized as having antiemetic properties and has been successfully used to treat nausea and vomiting in cancer patients undergoing chemotherapy. Cannabis has also been used in treating the weight loss syndrome of AIDS and for treating glaucoma by reducing intraocular pressure. Cannabis is also known for its muscle-relaxing and anti-convulsant effects.

The most prevalent mode of administration of medicinal Cannabis is by smoking. This mode of administration can have adverse effects on the lungs. Cannabis smoke carries more tar and other particulate matter than tobacco and may be a cause of lung diseases including lung cancer. Furthermore, many patients find the act of smoking unappealing, as well as generally unhealthy.

Accordingly, there is significant interest in developing other means to administer Cannabis to patients.

U.S. Patent Publication No. 2012/0231083 discloses an oral formulation of Cannabis useful for treating sleep apnea.

U.S. Patent Publication No. 2015/0057342 discloses an oral formulation of Cannabis compounds that provide an immediate release, a sustained release, and a combination of a sustained and immediate release through an oil-based cannabinoid formulation. These oral formulations required an additional banding step to prevent oil leakage from the capsules with an increased production cost.

To date, no easily divisible tablet exists for Cannabis. There remains an unmet need for an easily divisible tablet form of Cannabis or cannabinoid.

Divisible pharmaceutical tablets generally allow a pharmaceutical composition to be provided to patients in a measured, predetermined dose. Should the patient be prescribed to, or otherwise desire to, sub-divide the predetermined dose into one or more smaller doses, he or she can break or fracture the divisible tablet along one or more score lines to form individual fragments or sub-portions of the tablet. Each sub-portion generally includes a fractional amount of pharmaceutical; with tablets often being divisible into equal portions, such as, for example, half portions, third portions, or quarter portions.

### SUMMARY OF THE INVENTION

In one aspect, the invention provides a scored cannabinoid tablet composition, the tablet comprising: an upper surface, a lower surface, and a peripheral side surface, wherein each of the upper and lower surfaces forms an arcuate elevating from the peripheral edge toward the center thereof; at least one V-shaped groove as a score line on the upper surface, wherein an inner angle of the V shape of the V-shaped groove ranges from about 50° to about 90° and the depth of the V-shaped groove in the center of the upper surface ranges from about ⅓ to about ⅛ of the height of the peripheral side surface; and wherein the tablet comprises at least one cannabinoid extract from a Cannabis plant. In some embodiments, the tablet further comprises a microcrystalline cellulose, a medium chain triglyceride, a polysorbate, or a combination thereof.

In another aspect, the invention provides a method of treating a disease or disorder, the method comprising administering a scored cannabinoid tablet composition of the invention.

In yet another aspect, the invention provides a method of manufacturing a scored cannabinoid tablet composition, the method comprising the steps of: providing least one cannabinoid extract from a Cannabis plant; combining the extract with a pharmaceutically acceptable carrier in a solvent to form granules using a high shear granulation means; drying the granules; blending the dried granules with sodium starch glycolate to form a powder blend; and hard pressing the powder blend into tablets.

Other features and advantages of the present invention will become apparent from the following detailed description, examples, and figures. It should be understood, however, that the detailed description and the specific examples, while indicating embodiments of the invention, are given by way of illustration only since various changes and modifications within the spirit and scope of the invention will become apparent to those skilled in the art from this detailed description.

### BRIEF DESCRIPTION OF THE FIGURES

The present invention will be more fully understood with reference to the following detailed description which is accompanied by drawings.

FIG. 1 shows a top view of a tablet, according to one embodiment of the invention.

FIG. 2 shows a side view of a tablet, according to one embodiment of the invention.

### DETAILED DESCRIPTION OF THE INVENTION

Before the present invention is disclosed and described, it is to be understood that this invention is not limited to the particular structures, process steps, or materials disclosed herein, but is extended to equivalents thereof as would be recognized by those of ordinary skill in the relevant arts. It should also be understood that the terminology employed herein is used for the purpose of describing particular embodiments only and is not intended to be limiting.

### Tablets

As illustrated generally in the attached figures, in one aspect of the invention, a divisible pharmaceutical tablet is provided. The tablet can be used to orally administer a cannabinoid composition to a patient. The tablet can be formed from a variety of materials known to those of ordinary skill in the art. The composition incorporates, in part, a formulation for the release of a cannabinoid in order to avoid problems inherent with oil-based capsules.

In one embodiment, the invention relates to a scored tablet for a multiple dosing, wherein said tablet is capable of being easily broken into two or more parts, for example by holding the tablet between the thumb and forefinger and applying the requisite compressive breaking force.

In one embodiment, the tablet of the invention has an upper surface, a lower surface, and a peripheral side surface disposed therebetween. One or both of the upper and lower surfaces can form, for example, a curve or an arcuate elevating from the peripheral edge toward the center of the tablet. The curvature of the arcuate upper and/or lower surfaces can trace a simple arc in which the elevation level changes from one edge to the other edge through a peak at a constant radius of curvature. Alternatively, curvature of the arcuate upper and/or lower surfaces such that the surface is elevated sharply in edge regions at a small radius of curvature and the elevated manner changes in a peak region to a large radius of curvature (gentle curve). In another alternative, the upper and/or lower surfaces have a complex shape wherein the surface is elevated linearly in edge regions and the elevated manner changes in a peak region to a large radius of curvature. The present invention also contemplates the upper and lower surfaces having the same or different curvatures.

The peripheral side surface disposed between the upper and the lower surfaces in the tablet according to the invention may be flat, curved, or have a more complex shape. In one embodiment, the peripheral side surface is convex. In another embodiment, the peripheral side surface is concave.

The term “convex,” as used herein, refers to the state of a surface or a line as generally being curved or rounded outwardly. It is to be understood that, when the term convex is used herein to describe a property of a surface, the surface being referenced is convex in a three-dimensional space. That is, portions of a convex surface slope away from and downward relative to an apex of the surface, from all directions from the apex (although different segments of the surface may slope to a greater or lesser degree than other segments). As an example, the outer surface of a typical optical contact lens would be considered a convex surface, as that term is used herein.

The term “concave,” as used herein, refers to the state of a surface or a line as generally being curved or rounded inwardly. It is to be understood that, when the term concave is used herein to describe a property of a surface, the surface being referenced is concave in a three-dimensional space. That is, portions of a concave surface slope upward relative to a trough of the surface, from all directions from the trough (although different segments of the surface may slope to a greater or lesser degree than other segments). As an example, the inner surface of a bowl would be considered a concave surface, as that term is used herein.

The tablets of the invention further comprise at least one groove allowing for the tablet to be split into at least two factional portions. In one embodiment, the one or more grooves span only the upper surface of the tablet. In another embodiment, the one or more grooves span only the lower surface of the tablet. In a further embodiment, the one or more grooves span both the upper and the lower surface of the tablet. In a yet further embodiment, the one or more grooves span the entire circumference of the tablet according to the present invention, i.e. the upper surface, the lower surface and the peripheral side surface.

The tablets of the present invention may have a variety of shapes, including, without limitation, generally round shapes, “caplet” shapes, and polyhedral shapes (e.g., triangular, rectangular, etc.). In one embodiment, the tablets according to present invention are circular. In another embodiment, the tablets according to present invention have an oblong shape, for example an oval shape, to facilitate relatively effortless deglutition by patients. As contemplated in the present invention, examples of the oval shape include an oval shape having two straight-line portions and two semicircular arc portions, a mathematically defined elliptic curve, and an approximately oval shape comprising two arc portions with a small radius and two arc portions with a large radius and interposed therebetween and smoothly joined with one another. The shape of the individual segments and whole tablet, as well as the depth and angle of the dividing grooves, may be varied depending on the desired dose per tablet; tablet size, weight, strength and other formulation considerations.

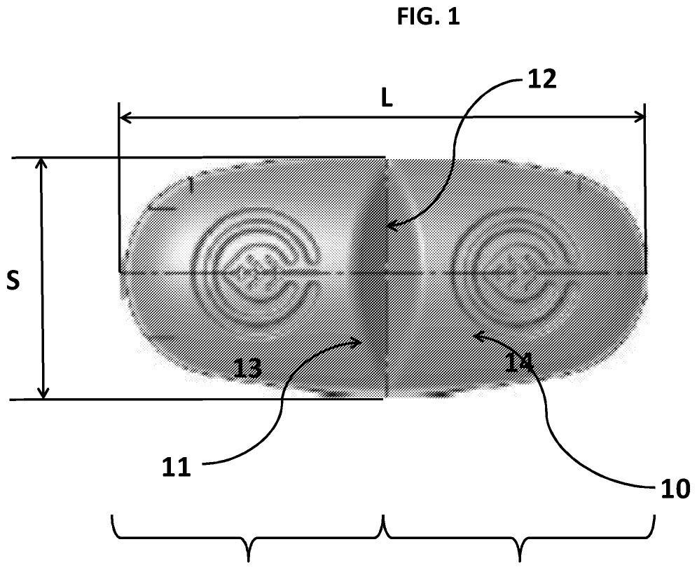

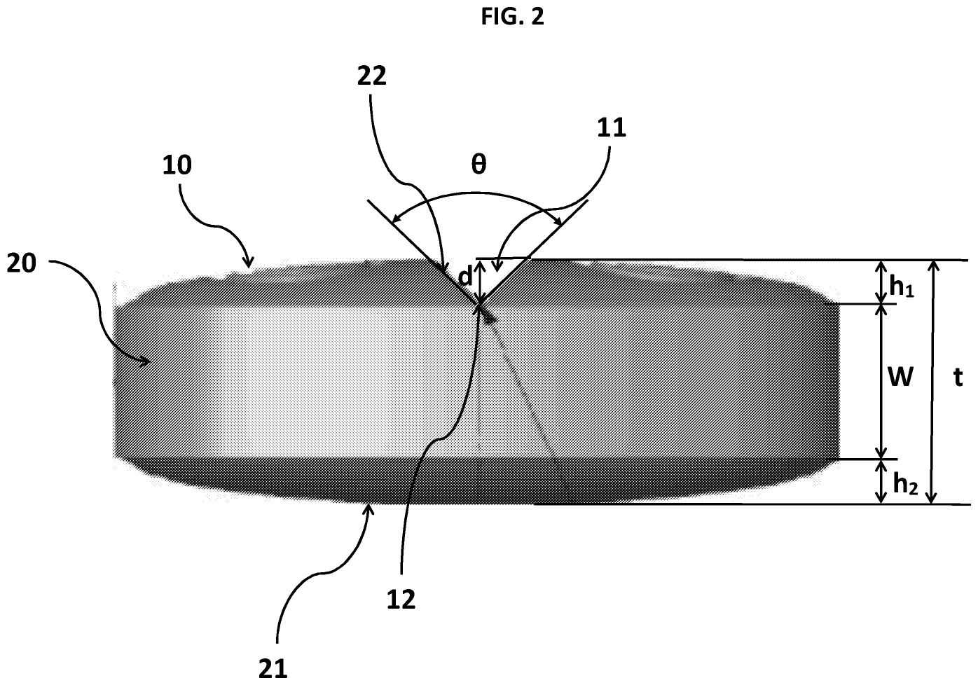

FIG. 1 and FIG. 2 show a schematic structure of one embodiment of the scored tablet. As shown in FIG. 1, the scored tablet has a generally oval shape having a major axis L and a minor axis S. As shown in FIG. 2, the tablet has an upper surface 10, a lower surface 21, and a peripheral side surface 20 having thickness W disposed therebetween. The upper and the lower surfaces are convex, each surface rising to the apex with an elevation h₁ (upper surface) or h₂ (lower surface) with both apices located at the intersection of the major and minor axis of the oval. As one of the ordinary skill on the art would recognize, when the upper surface and lower surface have same curvature h₁=h₂, and when curvatures are different h₁ and h₂ will have different values. In a further aspect, the embodiment of the invention includes a dividing groove 11 having lateral walls 22 and the bottom 12, aligned with the minor axis of the oval, and allowing the tablet to be easily split along the minor axis of the oval into two segments 13 and 14. The cross-sectional shape of groove 11 is an isosceles triangle (where the lateral walls 22 form the legs of the triangle) and the depth of the groove varies due to the curved shape of the upper surface 10, being most shallow at the lateral edges of the groove and reaching maximum depth at the geometric center of the tablet. As contemplated in the present invention, the inner angle θ between lateral groove walls ranges from about 50° to about 90°, as well as a value of 70°.

As used herein, the term “apex” is to be understood to refer to a portion of a curvature (whether or not the curvature is formed by a line or a surface) from which adjacent portions of the curvature slope away. In other words, the apex forms the “highest” portion of a curvature, the portion above which no other portion of the curvature rises. It is to be understood that, as used herein, the term apex may refer to a single point in space or may refer to a collection of generally co-planar points in space. Thus, a small, flat section of a surface can comprise an apex, as can a sharply peaked portion (e.g., a single point) of the surface.

The present invention encompasses dividing grooves having different form and shape. In one embodiment, the dividing groove is V-shaped. In another embodiment, the dividing groove is U-shaped. It is to be noted that the “V-shaped groove” includes a substantially V-shaped groove. The “substantially V-shaped groove” includes a case in which the vicinity of the bottom of the V-shaped groove has a shape formed so as to be round with a curved line or is a shape similar to the vicinity of the vertex of a parabola (quadratic curve), a case in which the vicinity of the bottom of the V-shaped groove is a polygon shape having a bended part and the like. Likewise, the U-shaped groove includes a substantially U-shaped groove. The “substantially U-shaped groove” includes, for example, a case in which a round curved line portion of the U-shaped groove is formed into a polygon shape having a bended part and the like. The present invention further contemplates grooves having lateral walls that are planar, convex, or concave.

The invention additionally contemplates dividing grooves having different bottom profiles. In one embodiment, the bottom of one or more dividing grooves (designated 12 in the embodiments shown in FIG. 1 and FIG. 2) is a straight line, e.g., perpendicular to peripheral side walls. In another embodiment, the bottom of the dividing groove may be elevated more on the center than on the peripheral edges of the upper or lower surface. Thus, in one embodiment, part of the bottom of the V-shaped groove is in the form of a simple arc with a constant radius of curvature throughout the entire length. In another embodiment, the bottom of the V-shaped groove is linearly elevated from both edges toward the center part of the groove, wherein the center part may be either one point peak, or a horizontal linear portion. In another embodiment, the bottom of one or more dividing grooves may be depressed more on the center than on the peripheral edges of the upper or lower surface. Such a depressed groove bottom, similarly to an elevated groove bottom, may have a simple shape like an arc or a more complex shape.

The present invention also contemplates tablets having dividing grooves having different depths relative to overall thickness of the tablet. In one embodiment, the ratio of maximum depth of the groove d to the elevation h of the curved surface (d/h) ranges from about 0.4 to about 1.0 (see FIG. 2). In another embodiment, the ratio of the maximum depth of the groove d to the total thickness t of the tablet (d/t) ranges from about 0.1 to about 0.5. In another embodiment, the ratio of the maximum depth of the groove d to the height W of the peripheral side surface (d/W) 0.125 (i.e., ⅛) to about 0.33 (i.e., ⅓).

The present invention further encompasses tablets having more than one dividing groove, allowing a consumer to divide the tablet into three or more sub-portions and thus creating incremental, fractional dosages for use by the consumer. Depending upon the composition of the tablet, one or more of the fractional dosages can be used by the consumer at the time of splitting, and the remaining portions can be saved for use at a later time. The grooves may have parallel arrangement, or two or more grooves may intersect at an angle ranging from 10° to 90°, thus allowing each fractional portion to be further subdivided into smaller segments. Thus, for example, an oval tablet may have the first dividing groove aligned with the major axis, a second dividing groove aligned with the minor axis, and third and fourth dividing grooves extending across the tablet at an oblique angle relative to both axes. In other embodiments, location, orientation, configuration, and number of dividing grooves can be varied along with the shape of the tablet to provide the most effective divisible tablet for the pharmaceutical agent being delivered.

The dividing grooves can create a zone of weakness in the tablet sufficient to facilitate division of the tablet through reducing the force required for breaking the scored tablet (“bending strength”). In one embodiment, the required force ranges from about 10 N to about 50 N. In another embodiment, the required force ranges from about 10 N to about 40 N. The bending strength varies depending on, for example, the external shape of the scored tablet, the cross-sectional area appearing when the scored tablet is divided along the score line, the shape of the cross-section of the V-shaped groove serving as the score line, the composition and the content ratios of the contained components of the plain tablet, and the compression force. The combination of these parameters that would yield a desired bending strength can be easily determined using methods known in the art. Importantly, the scored tablets of the invention retain sufficient strength to prevent premature breakage of the tablet either during manufacture, insertion into a container, or during transit of the container to the end user.

When a consumer wishes to divide the tablet, she can apply a bending force to the tablet by, for example, applying force with her fingers and/or thumbs to opposing sides of the tablet adjacent the dividing notches, and thereby “break” the tablet into fragments.

While the examples shown in the accompanying figures include a tablet divisible into two segments of substantially equal volume, it is to be understood that tablets of the present invention can be divisible into a number of different fractional pieces, including, without limitation, 2 fractional pieces, 3 pieces, 4, 5, 6, 7, 8, etc.

In addition, while the segments shown are of substantially equal volume, it is to be understood that tablets of the present invention can be divisible into multiple segments that are not necessarily equal in volume. In one example (not shown), a tablet can be provided that can be divisible into three segments, with one segment having a volume approximately equal to the combined volume of the remaining two segments.

Regardless of the number, size, shape, or volume of the segments into which the present tablet can be divided, division thereof produces segments that consistently provide a desired dosage of the pharmaceutical agent being administered. In one aspect of the invention, the dosage uniformity provided by the individual segments can be dosage units of within about 85% to about 115% of the labeled amount indicated for each segment in accordance with the USP-NF dosage uniformity guidelines. In another aspect, the dosage uniformity provided may be within about 80% to about 120%, inclusive of sub-ranges within this span. In yet another aspect, the dosage uniformity may be from about 90% to about 110%. In a further aspect, the dosage uniformity may be from about 95% to about 105%. In another aspect, the dosage uniformity may be about 100%.

The scored tablet of the invention may be debossed or printed with letters for identification.

### Compositions

One embodiment of the invention relates to oral compositions of cannabinoids to provide a release of a combination of Tetrahydrocannabinol (THC) and Cannabidiol (CBD) in a powder form derived from a direct compression method utilizing microcrystalline cellulose as an excipient. The cannabinoids are easily prepared and formulated to provide consistent therapeutically effective dosage forms from lot to lot.

The inventors of the instant application unexpectedly discovered that a solid pill oral composition which includes, in part, an extracted cannabinoid combined with microcrystalline cellulose provides for the elimination of leaking as seen in oil-based capsules. Further the formulation disclosed herein reduces hallucinatory effects associated with large changes in dose.

In one embodiment, the invention relates to an oral composition, for example, a scored tablet of a cannabinoid, the tablet comprising one or more cannabinoids in a powder form derived from a direct compression method that utilizes microcrystalline cellulose as an excipient.

In some embodiments, the cannabinoid is a cannabinoid extract that contains a combination of at least two of the following: Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM) and derivatives thereof. The cannabinoid may be natural or synthetic.

The methods of making cannabinoids extract is well known in the art. The Cannabis plants are grown, harvested, and the cannabinoids are extracted through, for example, a CO₂ extraction process.

In some embodiments, the cannabinoids are in equal proportion such as 2.5 mg THC to 2.5 mg CBD. Other embodiments include proportions of THC/CBD of 0.25 mg THC to 5.0 mg CBD or 5.0 mg THC to 0.25 mg CBD per tablet.

In a formulation of the invention, the first cannabinoid can be present in an amount ranging from about 0.01 mg to about 200 mg. The second cannabinoid also can be present in an amount ranging from about 0.01 mg to about 200 mg.

In one embodiment, the microcrystalline cellulose is present in an amount ranging from about 1 mg to about 200 mg per tablet. In another embodiment, the microcrystalline cellulose is present in an amount ranging from about 100 mg to about 200 mg per tablet. In another embodiment, the microcrystalline cellulose is present in an amount ranging from about 140 mg to about 143 mg per tablet.

In one embodiment, the microcrystalline cellulose is present in the tablet is about 1% to about 99% by weight based on 100 parts by weight of the tablet. In another embodiment, the microcrystalline cellulose is present in the tablet is about 80% to about 99% by weight based on 100 parts by weight of the tablet. In another embodiment, the microcrystalline cellulose is present in the tablet is about 94% to about 95% by weight based on 100 parts by weight of the tablet.

In one embodiment, the sodium starch glycolate is present in an amount ranging from about 0.01 mg to about 200 mg per tablet. In another embodiment, the sodium starch glycolate is present in an amount ranging from about 0.01 mg to about 10 mg per tablet. In another embodiment, the sodium starch glycolate is present in an amount ranging from about 2 mg to about 4 mg per tablet.

In one embodiment, the sodium starch glycolate is present in the tablet is about 0.01% to about 99% by weight based on 100 parts by weight of the tablet. In another embodiment, the sodium starch glycolate is present in the tablet is about 0.01% to about 5% by weight based on 100 parts by weight of the tablet. In another embodiment, the sodium starch glycolate is present in the tablet is about 1% to about 3% by weight based on 100 parts by weight of the tablet.

The compositions disclosed herein include a composition for daily administration. In one embodiment, the therapeutic effect is maintained with one tablet, twice daily. In another embodiment, the therapeutic effect is maintained with one tablet segment, twice daily. The duration of each dose's effect is between four (4) to six (6) hours.

Examples of a disease or a disorder that can be treated by the invention include, but are not limited to, pain associated with cancer, neuropathic pain and HIV-associated sensory neuropathy, side effects of chemotherapy including nausea and pain, symptoms of neurological and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis, and dystonia.

In some embodiments where the disorder is cancer, pain associated with cancer, nausea associated with chemotherapy, or a combination thereof, the composition described herein exerts reduced hallucinatory effects compared to smoking a Cannabis containing cigarette or ingesting a Cannabis containing foodstuff with the same amount of active ingredients.

The scored tablets of the present invention may also contain additional ingredients such as solvents, carriers, or excipients.

In some embodiments, the solvent comprises ethanol, methanol, isopropanol, chloroform, propylene glycol, polyethylene glycol, glycerine, limonene, myrcene, linalool, alpha bisabolol, delta 3 carene, borneol, alpha-pinene, beta-pinene, eucalyptol, terpineol, caryophyllene, camphene, or combinations thereof.

In some embodiments, the carrier comprises cellulose, microcrystalline cellulose, silicified microcrystalline cellulose, starch, pregelatinized starch, dicalcium phosphate, tricalcium phosphate, or mixtures thereof. In other embodiments, the carrier may be comprised of a water-soluble sugar or sugar alcohol. Examples include, lactose, sucrose, dextrose, polydextrose, fructose, maltose, maltodextrin, dextrate, dextrin, lactitol, mannitol, erythritol, maltitol, sorbitol, or xylitol, and mixtures thereof.

In some embodiments, the cannabinoid of the invention is any member of a group of substances that are structurally related to tetrahydrocannabinol and that bind to a cannabinoid receptor such as CB1 or CB2 or both (‘THC’). The cannabinoid can be a naturally occurring compound (e.g., present in Cannabis), a compound metabolized by a plant or animal, or a synthetic derivative.

The cannabinoid may be included in its free form or in the form of a salt; an acid addition salt of an ester; an amide; an enantiomer; an isomer; a tautomer; a prodrug; a derivative of an active agent of the present invention; different isomeric forms (e.g., enantiomers and diastereoisomers), both in pure form and in admixture, including racemic mixtures; or in enol forms.

The cannabinoids of the invention are further meant to encompass natural cannabinoids, natural cannabinoids that have been purified or modified, and synthetically-derived cannabinoids. For example, United States Patent Application Publication No. 2005/0266108, which is hereby incorporated by reference in its entirety, describes a method of purifying cannabinoids obtained from plant material.

The cannabinoids of the invention can be any of 9-tetrahydrocannabinol, 8-tetrahydrocannabinol, (+)-1,1-dimethylheptyl analog of 7-hydroxy-delta-6-tetrahydrocannabinol, 3-(5′-cyano-1′,1′-dimethylpentyl)-1-(4-N-morpholinobutyryloxy) delta-8-tetrahydrocannabinol hydrochloride, dexanabinol, nabilone, levonantradol, or N-(2-hydroxyethyl)hexadecanoamide. In some embodiments, the cannabinoids of the invention can be any of the non-psychotropic cannabinoid 3-dimethylnepty 11 carboxylic acid homologine 8, delta-8-tetrahydrocannabinol.

In another embodiment, the scored tablets of the present invention further comprise a film coating. Materials for film coating include, but are not limited to, for example, coating base and coating ingredients. These materials are used in an amount which is commonly used in the pharmaceutics formulation area. Numerous types of film coating are described in the art and, for example, in U.S. Pat. No. 8,541,024, hereby incorporated by reference in its entirety. An example of a film forming polymer used to form the film coating includes, for example, a cellulose polymer. The cellulose polymer has a molecular weight of 7,500 to 120,000 and a viscosity of 1.5 to 20 mm²/s (25 C°). Cellulose polymers include, for example, cellulose ether polymer, hydroxylpropyl methylcellulose (HPMC), hydroxylpropyl cellulose, methylcellulose, or ethylcellulose. TC-5 (Japanese Pharmacoepia hydroxypropyl methylcellulose 2910), for example, is used as HPMC.

The scored tablet may further contain an ingredient commonly used in the pharmaceutical field. Examples of the ingredient include, but are not limited to, a diluent, a disintegrant, a binder, a lubricant, a colorant, a pH-adjusting agent, a surfactant, a stabilizing agent, a sour agent, a flavor, a glidant, and the like. These ingredients are used in an amount commonly used in the pharmaceutical field unless otherwise specifically stated.

Examples of the diluent include, but are not limited to, a sugar or a sugar alcohol such as lactose (for example, lactose monohydrate), fructose, glucose, mannitol or sorbitol; a starch such as corn starch, potato starch, wheat starch, rice starch, partially pregelatinized starch, pregelatinized starch or porous starch; microcrystalline cellulose; anhydrous calcium phosphate, precipitated calcium carbonate, calcium silicate, and the like.

In the scored tablet, the diluent is used in an amount to give a content of from 5% to 95% by weight based on 100 parts by weight of the scored tablet of the invention.

For example, the disintegrant may be carboxymethyl cellulose, carboxymethyl cellulose calcium, carboxymethyl starch sodium, croscarmellose sodium, carmellose calcium, crospovidone, low-substituted hydroxypropyl cellulose, hydroxypropyl starch, or the like is used. The used amount of the disintegrant is an amount to give a content of from 0.5 to 25 parts by weight based on 100 parts by weight of the scored tablet of the invention.

Examples of the binder include hydroxypropyl cellulose (for example, grade: L, SL, SSL (trade name); Nippon Soda Co., Ltd.), hydroxypropylmethyl cellulose (for example, hypromellose 2910 (for example, TC-5 (grade: MW, E, EW, R, RW) (trade name); Shin-Etsu Chemical Co., Ltd.)), polyvinylpyrrolidone (povidone), gum arabic and the like.

In the scored tablet, the binder is used in an amount to give a content of from 1% to 20% by weight based on 100 parts by weight of the scored tablet of the invention.

Examples of the lubricant include magnesium stearate, calcium stearate, talc, sucrose fatty acid ester, sodium stearyl fumarate, and the like. The used amount of the lubricant is an amount to give a content of from 0.5% to 2% by weight based on 100 parts by weight of the solid preparation.

Examples of the colorant include a food dye (for example, food yellow No. 5, food red No. 2, food blue No. 2), a food lake color, iron oxide red, iron oxide yellow and the like.

Examples of the pH-adjusting agent include citrate, phosphate, carbonate, tartrate, fumarate, acetate, and amino acid salt.

Examples of the surfactant include sodium lauryl sulfate, polysorbate 80, polyoxyethylene (160), polyoxypropylene (30) glycol, and the like.

Examples of the stabilizing agent include tocopherol, tetrasodium edetate, nicotinamide, a cyclodextrin, and the like.

Examples of the sour agent include ascorbic acid, citric acid, tartaric acid, and malic acid.

Examples of the flavor include menthol, Mentha oil, lemon oil, and vanillin.

Examples of the glidant include colloidal silicon dioxide, aqueous silicon dioxide, and talc.

The above ingredients may be used by combining two or more members at an appropriate ratio.

### Manufacturing

In yet another aspect, provided herein is a method for the preparation of a formulation of tablets containing a combination of cannabinoids.

The scored tablets of the present invention may be manufactured using any method known in the art. In one embodiment, the method of manufacturing comprises an indirect tableting method wherein active ingredients and various additives are granulated through either fluidized bed granulation, agitation granulation, rolling granulation, rolling fluidized granulation, or extruding granulation, and then mixed with a lubricant as desired to obtain a powder for tableting. In one embodiment, the method of manufacture of the scored tablets of the present invention comprises the steps of providing at least one cannabinoid extract from a Cannabis plant; combining the extract with a pharmaceutically acceptable carrier in a solvent to form granules using a high shear granulation means; drying the granules; blending the dried granules with sodium starch glycolate to form a powder blend, and forming the tablets. In another embodiment, the cannabinoid extract is combined with microcrystalline cellulose and ethanol in a high shear granulation process. The resulting granules are then allowed to dry to remove the solvent prior to forming the tablets.

The scored tablets of the present invention can be formed using any method known in the art. In one embodiment, the scored tablets are formed by hard pressing. In another embodiment, the scored tablets are formed with a tableting machine, such as a rotary tableting machine or a high-productivity single shot tableting machine. In one embodiment, a well-known direct tableting method is used wherein a mixture of active ingredients and various additives acts as a powder for tableting and is tableted with a tableting machine, for example, a rotary tableting machine or a high-productivity single shot tableting machine. When compression molding is used, lubricants may be applied prior to the punch and die step of tableting machine and then compression molding may be performed without mixing the lubricant with the granules in the indirect tableting method or the mixture in the direct tableting method.

All patents and literature references cited in the present specification are hereby incorporated by reference in their entirety.

The present invention will be specifically explained by way of an example, but this example is not intended to limit the present invention.

### Example 1

### Preparation of Oral Hard-Fill Formulations for the Immediate-Release of Cannabinoids

Formulations described above are shown in Table 1 and are prepared using the methods described herein.

This manufacturing process of each individual tablet has an unexpectedly small variation from tablet to tablet, making this tablet and method of manufacture ideal in a large production environment. Manufacture of individual tablets have a maximum variation between each tablet in its target dose of approximately five percent (5%).

TABLE 1
The cannabinoids formulations

| | 20:1 THC:CBD | | 1:1 THC:CBD | | 1:20 THC:CBD | |
|---|---|---|---|---|---|---|
| Material | % | mg/tablet | % | mg/tablet | % | mg/tablet |
| THC Cannabinoid Extract | 3.33 | 5.0 | 1.67 | 2.5 | 0.17 | 0.25 |
| CBD Cannabinoid Extract | 0.17 | 0.25 | 1.67 | 2.5 | 3.33 | 5.0 |
| Microcrystalline Cellulose | 94.5 | 141.75 | 94.66 | 142.0 | 94.5 | 141.75 |
| Sodium Starch Glycolate | 2.0 | 3.0 | 2.0 | 3.0 | 2.0 | 3.0 |
| Ethanol | | | | | | |
| Totals | 100 | 150 | 100 | 150 | 100 | 150 |

It is to be understood that while the present example relates the composition having certain THC/CBD ratios, other THC/CBD ratios (e.g., 1:10, 10:1, etc.) are also encompassed by the invention and can be easily prepared using methods known in the art.

Unless defined otherwise, all technical and scientific terms used herein have the same meanings as commonly understood by one of ordinary skill in the art.

Having described embodiments of the present invention with reference to the accompanying drawings, it is to be understood that the present invention is not limited to the above-mentioned embodiments and that various changes and modifications can be affected by one skilled in the art without departing from the spirit or scope of the present invention as defined in the appended claims.

What is claimed is:

1. A scored cannabinoid tablet composition, the tablet comprising:
an upper surface, a lower surface and a peripheral side surface, wherein each of the upper and lower surfaces forms an arcuate elevating from the peripheral edge toward the center thereof;
at least one V-shaped groove as a score line on the upper surface, wherein an inner angle of the V shape of the V-shaped groove ranges from about 50° to about 90° and the depth of the V-shaped groove in the center of the upper surface ranges from about ⅓ to about ⅛ of the height of the peripheral side surface; and
wherein the tablet comprises at least one cannabinoid extract from a Cannabis plant.

2. The scored tablet according to claim 1, wherein the tablet further comprises microcrystalline cellulose, sodium starch glycolate, a medium chain triglyceride, a polysorbate, or a combination thereof.

3. The scored tablet according to claim 1, wherein the tablet further comprises microcrystalline cellulose and sodium starch glycolate.

4. The scored tablet according to any one of claims 2-3, wherein said microcrystalline cellulose is present in an amount of ranging from about 94% to about 95% by weight.

5. The scored tablet according to any one of claims 2-4, wherein said microcrystalline cellulose is present in an amount ranging from about 140 mg to about 143 mg per tablet.

6. The scored tablet according to any one of claims 2-5, wherein said sodium starch glycolate is present in an amount of about 2% by weight.

7. The scored tablet according to any one of claims 2-6, wherein said sodium starch glycolate is present in an amount about 3 mg per tablet.

8. The scored tablet according to any one of claims 1-7, wherein the tablet has an oval shape.

9. The scored tablet according to claim 8, wherein the V-shaped groves are oriented along the minor axis of said oval shape.

10. The scored tablet according to any one of claims 1-9 having two V shaped groves.

11. The scored tablet according to any one of claims 1-9 having one V shaped grove.

12. The scored tablet according to any one of claims 1-11, wherein the inner angle of the V shape of the V-shaped groove is 70°.

13. The scored tablet according to any one of claims 1-12, wherein the bending strength represented by a force required for dividing the scored tablet when the force is applied thereto so as to divide the tablet at the score line is 50 N or less.

14. The scored tablet according to any one of claims 1-13, wherein the composition has a combination of at least two cannabinoids.

15. The scored tablet according to claim 14, wherein the at least two cannabinoids are selected from a group consisting of Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM) and derivatives thereof.

16. The composition of any one of claims 14-15, wherein said at least two cannabinoids are in a 1:1 proportion by weight.

17. The composition of any one of claims 14-15, wherein said at least two cannabinoids are in a 10:1 proportion by weight.

18. The composition of any one of claims 14-15, wherein said at least two cannabinoids are in a 20:1 proportion by weight.

19. The composition of any one of claims 14-18, wherein said at least two cannabinoids are THC and CBD.

20. The composition of any one of claims 1-19, wherein at least one cannabinoid is THC present in an amount ranging from about 0.1 mg to about 200 mg.

21. The composition of any one of claims 1-19, wherein at least one cannabinoid is CBD present in an amount ranging from about 0.01 mg to about 200 mg.

22. A method for treating a disease comprising administering a daily therapeutically effective amount of the composition according to any one of claims 1-21.

23. The method of claim 22, wherein the therapeutically effective amount is contained in a single score-defined segment of the tablet.

24. The method of claim 22, wherein the disease is selected from a group consisting of pain associated with cancer, neuropathic pain and HIV-associated sensory neuropathy, side effects of chemotherapy including nausea and pain, symptoms of neurological and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis and dystonia.

25. A method of manufacturing a tablet cannabinoid composition, the method comprising the steps of:
providing least one cannabinoid extract from a Cannabis plant;
combining the extract with a pharmaceutically acceptable carrier and a solvent to form granules using a high shear granulation means;
drying the granules;
blending the dried granules with sodium starch glycolate to form a powder blend; and
hard pressing the powder blend into tablets.

26. The method of claim 25, wherein the extract is obtained through a CO₂ extraction process.

27. The method of any one of claims 25-26, wherein the tablet comprises at least two cannabinoids.

28. The method of claim 27, wherein at least two cannabinoids are selected from a group consisting of Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM) and derivatives thereof.

29. The method of any one of claims 27-28, wherein at least two cannabinoids are combined in a ratio selected from a group consisting of 1:1, 10:1, and 20:1.

30. The method according to any one of claims 27-29, wherein said at least two cannabinoids are THC and CBD.

31. The method of any one of claims 25-30, wherein at least one cannabinoid is CBD in an amount ranging from about 0.01 mg to about 200 mg.

32. The method of any one of claims 25-31, wherein the pharmaceutically acceptable carrier is selected from a group consisting of cellulose, microcrystalline cellulose, silicified microcrystalline cellulose, starch, sodium starch glycolate, pregelatinized starch, dicalcium phosphate, tricalcium phosphate, and mixtures thereof.

33. The method of claim 32, wherein the pharmaceutically acceptable carrier consists of microcrystalline cellulose and sodium starch glycolate.

34. The method of any one of claims 25-33, wherein the solvent is selected from a group consisting of ethanol, methanol, isopropanol, chloroform, propylene glycol, polyethylene glycol, glycerine, limonene, myrcene, linalool, alpha bisabolol, delta 3 carene, borneol, alpha-pinene, beta-pinene, eucalyptol, terpineol, caryophyllene, camphene, or combinations thereof.

35. The method of claim 34, wherein the solvent is ethanol.

36. A method of treating a disease or disorder, the method comprising administering a scored cannabinoid tablet composition, the tablet comprising: an upper surface, a lower surface, and a peripheral side surface, wherein each of the upper and lower surfaces forms an arcuate elevating from the peripheral edge toward the center thereof; at least one V-shaped groove as a score line on the upper surface, wherein an inner angle of the V shape of the V-shaped groove ranges from about 50° to about 90° and the depth of the V-shaped groove in the center of the upper surface ranges from about ⅓ to about ⅛ of the height of the peripheral side surface; and wherein the tablet comprises at least one cannabinoid extract from a Cannabis plant.
